(19) United States
(12) Patent Application Publication
(10) Pub. No.: US 2005/0176668 A1
(43) Pub. Date: Aug. 11, 2005

(54) VPS35-based assays and methods for treating Alzheimer's disease

(76) Inventors: Scott A. Small, New York, NY (US); Tae-Wan Kim, Old Tappan, NJ (US)

Correspondence Address: COOPER & DUNHAM, LLP, 1185 AVENUE OF THE AMERICAS, NEW YORK, NY, 10036, US

(21) Appl. No.: 10/980,921

(22) Filed: Nov. 3, 2004

Related U.S. Application Data
(60) Provisional application No. 60/518,250, filed on Nov. 7, 2003.

(51) Int. Cl. A61K 48/00; C12Q 1/68

(52) U.S. Cl. 514/44; 435/6

(57) ABSTRACT

This invention provides a method for determining whether an agent causes a reduction in the expression of a retromer complex protein. This invention further provides a method for determining whether an agent causes a reduction in the activity of a retromer complex. This invention also provides a method for reducing the expression of a retromer complex protein in a cell. This invention provides a method for treating a subject afflicted with Alzheimer's disease. This invention further provides a pharmaceutical composition as well as an article of manufacture. Finally, this invention provides a method for identifying a potential pathogenic nucleic acid transcript with respect to a brain disorder.

This application claims priority of U.S. Ser. No. 60/518,250, filed Nov. 7, 2003, the contents of which are hereby incorporated by reference into this application.

Throughout this application, certain publications are referenced. Full citations for these publications, as well as additional related references, may be found immediately preceding the claims. The disclosures of these publications are hereby incorporated by reference into this application in order to more fully describe the state of the art as of the date of the invention described and claimed herein.

This invention was made with support under United States Government Grant Nos. AG08702 and AG00949 from the National Institutes of Health. Accordingly, the United States Government has certain rights in the subject invention.

### BACKGROUND OF THE INVENTION

In recent years, remarkable strides have been made toward understanding the mechanisms underlying Alzheimer's disease (AD). A unifying molecular theme has emerged from a range of studies, showing that a regional increase in the concentration of a-β peptide, a cleaved product of amyloid precursor protein (APP), is fundamental to AD pathogenesis (Hardy and Selkoe 2002). Characterizing the mutations that underlie the autosomal-dominant form of AD, and transgenically expressing these mutations in mice, has established a causal role for a-β peptide (Selkoe and Podlisny 2002). Although etiologically more complex, a number of correlational studies have implicated a-β peptide in the pathogenesis of sporadic AD as well (Lue, Kuo et al. 1999; McLean, Cherny et al. 1999; Wang, Dickson et al. 1999; Naslund, Haroutunian et al. 2000). Most compelling are the studies showing an elevation in a-β peptide in selectively vulnerable regions, such as the entorhinal cortex, where peptide levels best discriminate between patients and controls, and more importantly, correlate with cognitive and synaptic dysfunction (Lue, Kuo et al. 1999; Naslund, Haroutunian et al. 2000).

The biochemical mechanisms that cause an increase in a-β concentration in most autosomal-dominant forms of AD have been worked out, where, for example, mutations in APP or its overexpression leads to increased peptide production (Selkoe and Podlisny 2002). In contrast, the molecular pathways that cause the regional increase in a-β concentration in sporadic AD, the disease's most common form, remain unknown.

Profiling patterns of gene expression, using techniques like microarray, has emerged as a powerful approach in isolating molecular differences between healthy and diseased tissue (Gu, Rao et al. 2002; Pongrac, Middleton et al. 2002). The exploratory power of microarray is also its main analytic liability. Distinguishing meaningful from false-positive expression differences, which naturally occur with multiple comparisons, is one of the main analytic challenges when using microarray (Slonim 2002). This challenge is heightened when investigating disorders of the brain (Pongrac, Middleton et al. 2002). Typically, only subtle changes in gene expression are necessary to cause neuronal dysfunction, and so large expression differences cannot be relied on to isolate meaningful gene products (Wurmbach, Gonzalez-Maeso et al. 2002). Furthermore, because tissue is typically harvested from post-mortem samples, sources of noise, such as factors that influence expression levels irrelevant to the disease process, are likewise more extensive. These factors extend beyond the individual genetic and environmental differences that affect any comparison, but also include individual differences in the dying process and even differences in how tissue is handled after death (Harrison, Heath et al. 1995). Thus, low sources of signal and large sources of noise need to be considered when microarray is applied to explore disorders of the brain.

Alzheimer's disease (AD) begins in the hippocampal formation (Jacobs, Sano et al. 1995; Braak and Braak 1996; Gomez-Isla, Price et al. 1996), a structure made up of multiple interconnected subregions. Each hippocampal subregion expresses a unique molecular profile (Zhao, Lein et al. 2001), accounting for why the subregions are differentially vulnerable to mechanisms of dysfunction (Small 2001). Histological and brain imaging studies have shown that cells in the entorhinal cortex are most sensitive to AD (West, Coleman et al. 1994; Braak and Braak 1996; Gomez-Isla, Price et al. 1996; Small, Perera et al. 1999; de Leon, Convit et al. 2001; Small, Tsai et al. 2002), while cells in the dentate gyrus, a neighboring hippocampal subregion, are relatively resistant to disease. pathogenesis (West, Coleman et al. 1994, Braak and Braak 1996; Small, Tsai et al. 2002). Imaging studies have further suggested that while the function of the entorhinal cortex is uniformly diminished in AD patients, entorhinal function is stable across the age-span among healthy controls (Small, Tsai et al. 2002). Using this information, a hypothesis-driven model showing how a molecule involved in AD pathogenesis should behave, both spatially and temporally may be designed.

### SUMMARY OF THE INVENTION

This invention provides a method for determining whether an agent causes a reduction in the expression of a retromer complex protein comprising the steps of (a) contacting the agent with a eukaryotic cell under conditions which, in the absence of the agent, permit expression of the retromer complex. protein; (b) after a suitable period of time, determining the amount of expression in the cell of the retromer complex protein; and (c) comparing the amount of expression determined in step (b) with the amount of expression which occurs in the absence of the agent, whereby a reduced amount of expression in the presence of the agent indicates that the agent causes a reduction in the expression of the retromer complex protein.

This invention further provides a method for determining whether an agent causes a reduction in the activity of a retromer complex comprising the steps of (a) contacting the agent with a eukaryotic cell under conditions which, in the absence of the agent, permit activity of the retromer complex; (b) determining the amount of activity in the cell of the retromer complex; and (c) comparing the amount of activity determined in step (b) with the amount of activity which occurs in the absence of the agent, whereby a reduced amount of activity in the presence of the agent indicates that the agent causes a reduction in the activity of the retromer complex.

This invention also provides a method for reducing the expression of a retromer complex protein in a cell comprising introducing into the cell an agent which specifically interferes with the expression of the retromer complex protein in the cell.

This invention provides a method for treating a subject afflicted with Alzheimer's disease comprising administering to the subject a therapeutically effective amount of an agent which specifically interferes with the expression of the retromer complex protein in the cells of the subject's brain which express a-β peptide.

This invention further provides a pharmaceutical composition comprising an agent which specifically interferes with the expression of a retromer complex protein when introduced into a cell and a pharmaceutically acceptable carrier.

This invention also provides an article of manufacture comprising a packaging material having therein an agent which specifically interferes with the expression of a retromer complex protein when introduced into a cell and a label indicating a use for the agent in treating a subject afflicted with Alzheimer's disease.

Finally, this invention provides a method for identifying a potential pathogenic nucleic acid transcript with respect to a brain disorder comprising the steps of (a) contacting (i) a brain nucleic acid microarray with (ii) a nucleic acid sample from afflicted brain tissue, wherein the brain tissue is from a brain region expected to contain the pathogenic nucleic acid transcript, under conditions permitting nucleic acid hybridization; (b) determining the hybridization pattern resulting from step (a); and (c) comparing the hybridization pattern so determined with the hybridization pattern determined upon separately contacting the microarray with a nucleic acid sample from (i) afflicted brain tissue which is not from a brain region expected to contain the pathogenic nucleic acid transcript, (ii) non-afflicted brain tissue which is from a brain region expected to contain the pathogenic nucleic acid transcript, and (iii) non-afflicted brain tissue which is not from a brain region expected to contain the pathogenic nucleic acid transcript, wherein if a nucleic acid from the sample of step (a) is present in a greater amount at a particular locus in the hybridization pattern of step (b) than at that locus in any of the hybridization patterns in step (c), or is present in a lesser amount at a particular locus in the hybridization pattern of step (b) than at that locus in any of the hybridization patterns in step (c), it is a potential pathogenic nucleic acid transcript.

### BRIEF DESCRIPTION OF THE FIGURES

FIG. 1

The experimental design based on modeling the assumed behavior of a pathogenic molecule in Alzheimer's disease.

(A) Spatial component of the model.

Among the hippocampal subregions, cells within the entorhinal cortex (EC) are more vulnerable to Alzheimer's disease compared to cells in the dentate gyrus (DG).

(B) Constraining sources of noise.

The transcriptisome of the entorhinal cortex contains a pathogenic transcript, but is also influenced by sources irrelevant to the disease process, such as regionally specific expression, as well as individual genetic and environmental differences. A 2×2 factorial design can control for these between-group and between-region sources of noise, and isolate the pathogenic transcript.

(C) Temporal component of the model.

Expression levels of a pathogenic molecule should be reliably different when comparing between a group of AD patients (black triangles) and healthy controls (gray diamonds), but at the same time expression of the molecule should not differ across the age-span when assessing within a group of healthy controls (gray diamonds) . The temporal component of the model was used as a filter against false-positive findings.

FIG. 2A-B

Expression of only four genes conformed to the spatiotemporal model of Alzheimer's disease.

Although the factorial analysis was performed on the raw expression levels, for graphical purposes the data are shown as percent expression in the entorhinal cortex (EC expression—DG expression)/DG expression. Each point represents a single subject, and the subjects are separated into the two experimental groups. VPS35; calnuc, COP9, and proteosome subunit IV, conformed to both the temporal and as shown, the spatial components of the model.

FIG. 3

VPS35 is the single transcript that best discriminates Alzheimer's disease from controls.

By performing a stepwise logistic regression, VPS35 was isolated as the primary transcript associated with Alzheimer's disease. When included in this multivariate analysis, calnuc, COP9, and proteosome subunit IV were no longer associated with AD.

(A) Real-time PCR confirmed the VPS35 effect as detected with microarray.

(B) Immunocytochemical staining using anti-VPS35 antibody demonstrates that protein expression is neuronal predominant, as shown in the entorhinal cortex of an AD brain.

(C) An alternative representation of the data shows that the microarray effect was caused by a differential increase in entorhinal cortex expression, and not by a differential decrease in dentate gyrus expression.

(D) The same representation of the RT-PCR data shows a similar pattern.

FIG. 4

VPS35 is selectively associated with BACE and a sortilin-like receptor in the human hippocampal formation.

The associations were found by correlating VPS35 expression to the expression of any type I membrane-protein in the entorhinal cortex of AD brains (circles);

the correlations were preserved when both groups and both subregions were included in the analysis (stars).

(A) The linear correlation observed between VPS35 and BACE

(B) The linear correlation observed between VPS35 and SorLA

(C) No correlation was observed between BACE and SorLA, showing that they are independently correlated with VPS35.

FIG. 5

The expression of BACE, APP, and SorLA do not conform to the spatial model of Alzheimer's disease.

FIG. 6

A model showing how VPS35 can contribute to the pathogenesis of sporadic Alzheimer's disease.

(A) Normal trafficking of type I membrane-proteins. Once expressed in the nucleus (nuc) and modified in the endoplasmic reticulum (ER), type I membrane-proteins ( [symbol] ), like BACE, are typically trafficked among the trans-golgi network (TGN), cell surface, and endosome, and then degraded in the lysosome.

(B) Accumulation of type I membrane-proteins in the endosome and trang-golgi network. Overexpressing VPS35 leads to increased concentrations of type I membrane-proteins in the endosome and TGN, either by decreasing lysosomal degradation or by increasing cell surface internalization. The endosome and the TGN are the two main traffic sites in which BACE cleaves amyloid-precursor protein (APP), leading to a-β production. By causing an accumulation of BACE in the endosome and TGN, VPS35 can indirectly accelerate the rate of a-β production.

### DETAILED DESCRIPTION OF THE INVENTION

Terms

As used in this application, except as otherwise expressly provided herein, each of the following terms shall have the meaning set forth below.

As used herein, “administering” an agent can be effected or performed using any of the various methods and delivery systems known to those skilled in the art. The administering can be performed, for example, intravenously, via cerebrospinal fluid, orally, nasally, via implant, transmucosally, transdermally, intramuscularly, and subcutaneously.

As used herein, “agent” shall mean any chemical entity, including, without limitation, a protein, an antibody, a nucleic acid, a small molecule, and any combination thereof.

As used herein, “antibody” shall include, by way of example, both naturally occurring and non-naturally occurring antibodies. Specifically, this term includes polyclonal and monoclonal antibodies, and antigen-binding fragments (e.g., Fab fragments) thereof. Furthermore, this term includes chimeric antibodies (e.g., humanized antibodies) and wholly synthetic antibodies, and antigen-binding fragments thereof.

As used herein, “antisense molecule” shall mean any nucleic acid which, when introduced into a cell (directly or via expression of another nucleic acid directly introduced into the cell), specifically hybridizes to at least a portion of an mRNA in the cell encoding a protein (i.e., target protein) whose expression is to be inhibited, and thereby inhibits the target protein's expression.

As used herein, a “brain disorder” shall mean any disorder or disease of the brain (e.g. Alzheimer's disease).

As used herein, “DNAzyme” shall mean a catalytic nucleic acid that is DNA or whose catalytic component is DNA, and which specifically recognizes and cleaves a distinct target nucleic acid sequence, which can be either DNA or RNA. Each DNAzyme has a catalytic component (also referred to as a “catalytic domain”) and a target sequence-binding component consisting of two binding domains, one on either side of the catalytic domain.

As used herein, “hybridization” shall mean the joining of two complementary strands of DNA or one each of DNA and RNA to form a double-stranded molecule. Hybridizing conditions are conditions permitting hybridization between two complementary strands of nucleic acids. Hybridizing conditions are well known in the art, and include, without limitation, physiological conditions. See Sambrook et al., 1989, “Molecular Cloning, A Laboratory Manual”, second edition, Cold Spring Harbor Press, Plainview, N.Y.

As used herein, “locus” shall mean a point, region or area for the affixation of a nucleic acid that does not overlap with another such point, region or area, and which may further be separated from another such point, region or area by physical space.

As used herein, “microarray” shall mean (a) a solid support having one or more compounds affixed to its surface at discrete loci, or (b) a plurality of solid supports, each support having one or a plurality of compounds affixed to its surface at discrete loci. The instant microarrays can contain all possible permutations of compounds within the parameters of this invention. For example, the instant microarray can be a disease-specific microarray, a species-specific microarray, or a tissue-specific microarray.

As used herein, “pharmaceutically acceptable carrier” shall mean any of the various carriers known to those skilled in the art.

The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

As used herein, “pathogenic nucleic acid transcript” shall mean a nucleic acid transcript (i.e. mRNA) whose altered expression plays a role in the onset and/or progression of a brain disorder. A “potential” pathogenic nucleic acid transcript is a transcript which may be, but has not yet been shown to be, a pathogenic nucleic acid transcript.

As used herein, a “brain region” shall mean any discrete physical portion of the brain.

As used herein, “retromer complex” shall mean a complex of proteins, wherein this complex (a) comprises a single VPS35 protein and one or more other proteins, and (b) performs functions including, for example, trafficking type I membrane proteins and acting to increase the concentration of membrane proteins in the endosome and trans-golgi network. “Retromer complex protein” shall mean one of the proteins which constitute a retromer complex.

As used herein, “ribozyme” shall mean a catalytic nucleic acid molecule which is RNA or whose catalytic component is RNA, and which specifically recognizes and cleaves a distinct target nucleic acid sequence, which can be either DNA or RNA. Each ribozyme has a catalytic component (also referred to as a “catalytic domain”) and a target sequence-binding component consisting of two binding domains, one on either side of the catalytic domain.

As used herein, “small interfering RNA” (also referred to as siRNA or RNAi) includes, without limitation, a polynucleotide sequence identical or homologous to a target gene (or fragment thereof) linked directly, or indirectly, to a polynucleotide sequence complementary to the sequence of the target gene (or fragment thereof). The siRNA optionally comprises a polynucleotide linker sequence of sufficient length to allow for the two polynucleotide sequences to fold over and hybridize to each other. The linker sequence is designed to separate the antisense and sense strands of siRNA significantly enough to limit the effects of steric hindrances and allow for the formation of a dsRNA molecule, and not to hybridize with sequences within the hybridizing portions of the dsRNA molecule. siRNA is discussed, e.g., in U.S. Pat. No. 6,544,783).

As used herein, “subject” shall mean any animal, such as a human, non-human primate, mouse, rat, guinea pig or rabbit.

As used herein, “suitable period of time” shall mean, with respect to the instant hybridization-based assay method, an amount of time sufficient to permit hybridization between two nucleic acids.

As used herein, “therapeutically effective amount” means an amount sufficient to treat a subject afflicted with a disorder or a complication associated with a disorder.

As used herein, “treating” a disorder shall mean slowing, stopping or reversing the disorder's progression.

### Embodiments of the Invention

This invention provides a method for determining whether an agent causes a reduction in the expression of a retromer complex protein, comprising the steps of (a) contacting the agent with a eukaryotic cell under conditions which, in the absence of the agent, permit expression of the retromer complex protein; (b) after a suitable period of time, determining the amount of expression in the cell of the retromer complex protein; and (c) comparing the amount of expression determined in step (b) with the amount of expression which occurs in the absence of the agent, whereby a reduced amount of expression in the presence of the agent indicates that the agent causes a reduction in the expression of the retromer complex protein. In the preferred embodiment of the instant method, the retromer complex protein is VPS35 (GenBank Accession No. BC002414for human VPS35).

In one embodiment of the instant method, the retromer complex protein is selected from the group consisting of VPS17 (GenBank Accession No. NC00147 for yeast VPS17), VPS26 (GenBank Accession No. BC022505 for human VPS26), VPS29 (GenBank Accession No. BC000880 for human VPS29), sorting nexin 1 (GenBank Accession No. AF065483 for human sorting nexin 1) and sorting nexin 2 (GenBank Accession No. AF065482 for human sorting nexin 2). In another embodiment, the cell is present in a cell culture. In another embodiment, the cell is a brain cell.

In one embodiment, determining the amount of expression is performed by determining the amount of retromer complex protein-encoding mRNA in the cell. In another embodiment, determining the amount of expression is performed by determining the amount of retromer complex protein in the cell. In a further embodiment, determining the amount of retromer complex protein in the cell is performed using an antibody specific for such protein.

This invention further provides a method for determining whether an agent causes a reduction in the activity of a retromer complex, comprising the steps of (a) contacting the agent with a eukaryotic cell under conditions which, in the absence of the agent, permit activity of the retromer complex; (b) determining the amount of activity in the cell of the retromer complex; and (c) comparing the amount of activity determined in step (b) with the amount of activity which occurs in the absence of the agent, whereby a reduced amount of activity in the presence of the agent indicates that the agent causes a reduction in the activity of the retromer complex. Activity of a retromer complex includes, for example (a) binding to a cargo moiety (e.g. sortelin-like receptor), (b) trafficking type I membrane proteins and (c) acting to increase the concentration of membrane proteins in the endosome and trans-golgi network. In the preferred embodiment of the instant method, the retromer complex protein is VPS35.

In one embodiment of the instant method, the retromer complex protein is selected from the group consisting of VPS17, VPS26, VPS29, sorting nexin 1 and sorting nexin 2. In another embodiment, the cell is present in a cell culture. In another embodiment, the cell is a brain cell.

This invention also provides a method for reducing the expression of a retromer complex protein in a cell comprising introducing into the cell an agent which specifically interferes with the expression of the retromer complex protein in the cell. In the preferred embodiment of the instant method, the retromer complex protein is VPS35.

In one embodiment of the instant method, the retromer complex protein is selected from the group consisting of VPS17, VPS26, VPS29, sorting nexin 1 and sorting nexin 2. In another embodiment, the cell is present in a cell culture. In another embodiment, the cell is a brain cell.

In one embodiment, the agent is a nucleic acid. In another embodiment, the nucleic acid is a small interfering RNA (siRNA). In further embodiments, the siRNA, whose target sequence is AAGTGGCAGATCTCTACGAAC, comprises either a sense siRNA having the sequence 5′-GUGGCAGAUCUCUACGAACdTdT or an antisense siRNA having the sequence 5′-GUUCGUAGAGAUCUGCCACdTdT. In further embodiments, the siRNA, whose target sequence is AAGCACAGCTAGCTGCCATCA, comprises either a sense siRNA having the sequence 5′-GCACAGCUAGCUGCCAUCAdTdT or an antisense siRNA having the sequence 5′-UGAUGGCAGCUAGCUGUGCdTdT. In other embodiments, the nucleic acid is a ribozyme, a DNAzyme or an antisense molecule.

This invention provides a method for treating a subject afflicted with Alzheimer's disease comprising administering to the subject a therapeutically effective amount of an agent which specifically interferes with the expression of the retromer complex protein in the cells of the subject's brain which express a-β peptide. In the preferred embodiment of the instant method, the retromer complex protein is VPS35.

In one embodiment of the instant method, the retromer complex protein is selected from the group consisting of VPS17, VPS26, VPS29, sorting nexin 1 and sorting nexin 2. In another embodiment, the cell is present in a cell culture. In another embodiment, the cell is a brain cell.

In one embodiment, the agent is a nucleic acid. In another embodiment, the nucleic acid is a small interfering RNA (siRNA). In further embodiments, the siRNA, whose target sequence is AAGTGGCAGATCTCTACGAAC, comprises either a sense siRNA having the sequence 5′-GUGGCAGAUCUCUACGAACdTdT or an antisense siRNA having the sequence 5′-GUUCGUAGAGAUCUGCCACdTdT. In further embodiments, the siRNA, whose target sequence is AAGCACAGCTAGCTGCCATCA, comprises either a sense siRNA having the sequence 5′-GCACAGCUAGCUGCCAUCAdTdT or an antisense siRNA having the sequence 5′-UGAUGGCAGCUAGCUGUGCdTdT. In other embodiments, the nucleic acid is a ribozyme, a DNAzyme or an antisense molecule.

This invention further provides a pharmaceutical composition comprising an agent which specifically interferes with the expression of a retromer complex protein when introduced into a cell; and a pharmaceutically acceptable carrier.

This invention also provides an article of manufacture comprising a packaging material having therein an agent which specifically interferes with the expression of a retromer complex protein when introduced into a cell and a label indicating a use for the agent in treating a subject afflicted with Alzheimer's disease.

Finally, this invention provides a method for identifying a potential pathogenic nucleic acid transcript with respect to a brain disorder comprising the steps of (a) contacting (i) a brain nucleic acid microarray with (ii) a nucleic acid sample from afflicted brain tissue, wherein the brain tissue is from a brain region expected to contain the pathogenic nucleic acid transcript, under conditions permitting nucleic acid hybridization; (b) determining the hybridization pattern resulting from step (a); and (c) comparing the hybridization pattern so determined with the hybridization pattern determined upon separately contacting the microarray with a nucleic acid sample from (i) afflicted brain tissue which is not from a brain region expected to contain the pathogenic nucleic acid transcript, (ii) non-afflicted brain tissue which is from a brain region expected to contain the pathogenic nucleic acid transcript, and (iii) non-afflicted brain tissue which is not from a brain region expected to contain the pathogenic nucleic acid transcript, wherein if a nucleic acid from the sample of step (a) is present in a greater amount at a particular locus in the hybridization pattern of step (b) than at that locus in any of the hybridization patterns in step (c), or is present in a lesser amount at a particular locus in the hybridization pattern of step (b) than at that locus in any of the hybridization patterns in step (c), it is a potential pathogenic nucleic acid transcript. In one embodiment, the instant method further comprises the step of determining whether the increase or decrease, as applicable, in the amount of potential pathogenic nucleic acid transcript so identified is concurrent with the onset of the brain disorder in an afflicted subject. In the preferred embodiment, the subject is human. In a further embodiment, the brain disorder is Alzheimer's disease.

This invention is illustrated in the Experimental Details section which follows. This section is set forth to aid in an understanding of the invention but is not intended to, and should not be construed to limit in any way the invention as set forth in the claims which follow thereafter.

### EXPERIMENTAL DETAILS

Introduction

Although many of the molecules causing the rare form of familial Alzheimer's disease (AD) have been identified, the molecules responsible for sporadic AD, which accounts for over 95% of all cases of AD, remain unknown. This study relies on brain imaging findings to model the manner in which a pathogenic molecule should behave, spatially and over time. When applied to microarray data, the model isolated a trafficking molecule whose expression best discriminated sporadic AD from controls. The identified molecule, VPS35, is the core component of a sorting complex that traffics type I membrane-proteins, and acts to increase the concentration of trafficked proteins in the endosome and trans-golgi network. On further analysis, the expression of only two type I membrane-proteins in the human hippocampal formation were associated with VPS35—one of which was unexpectedly β-site APP-cleaving enzyme (BACE). BACE is responsible for producing a-β peptide, the key factor underlying AD pathophysiology, and the enzyme's activity is optimized within the endosome and trans-golgi network. Taken together, these findings identify a membrane trafficking pathway that can account for differential a-β production and AD pathogenesis.

Synopsis

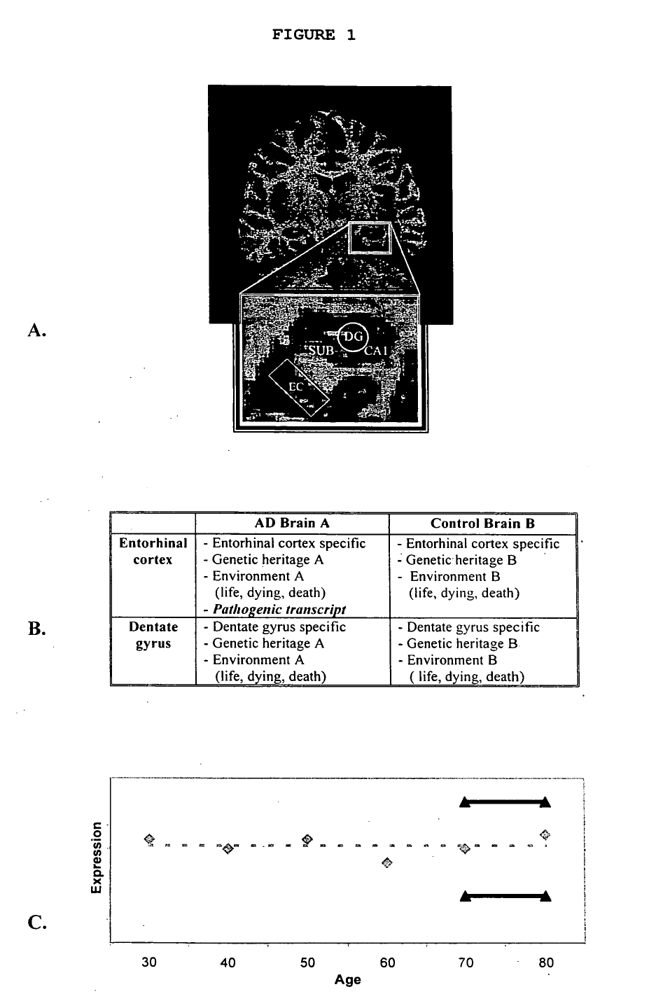

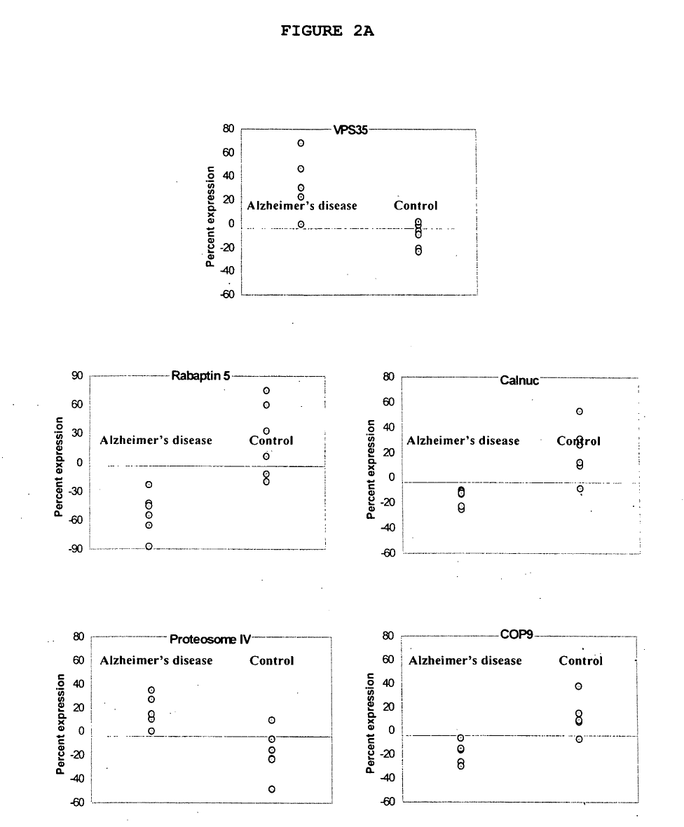

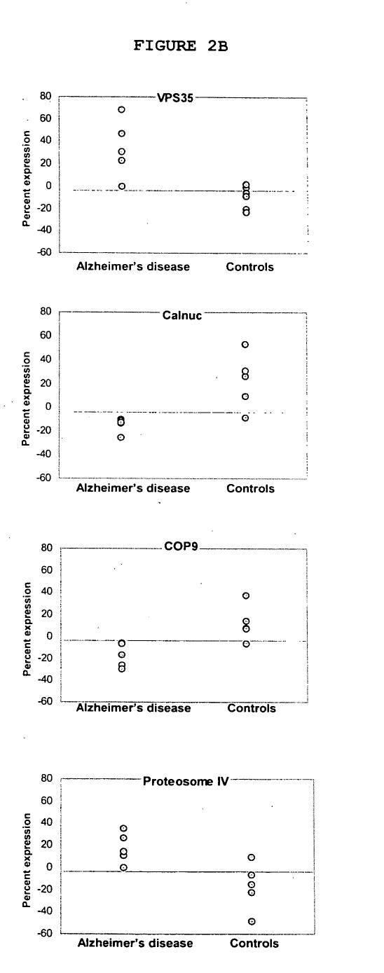

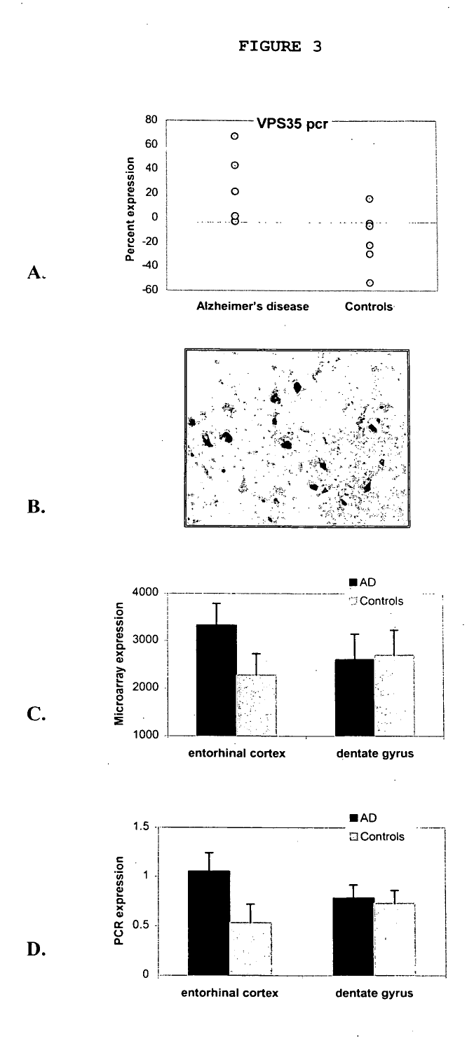

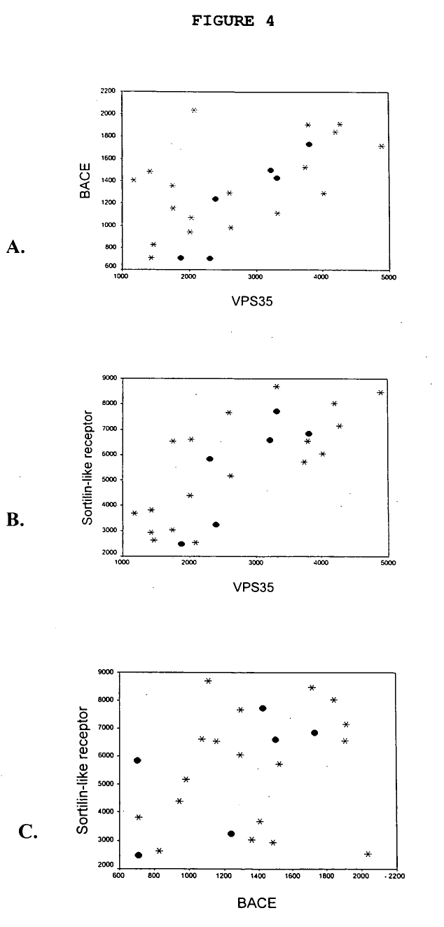

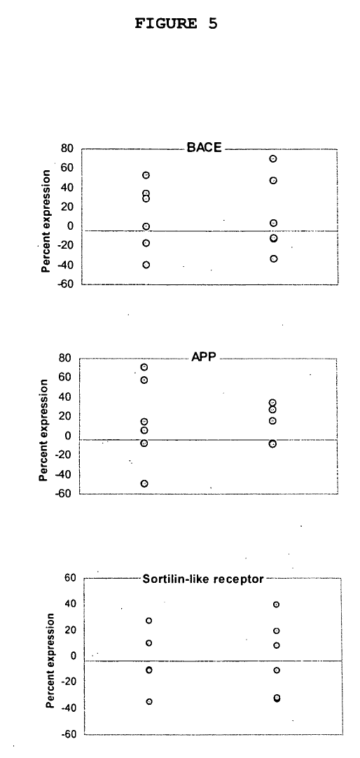

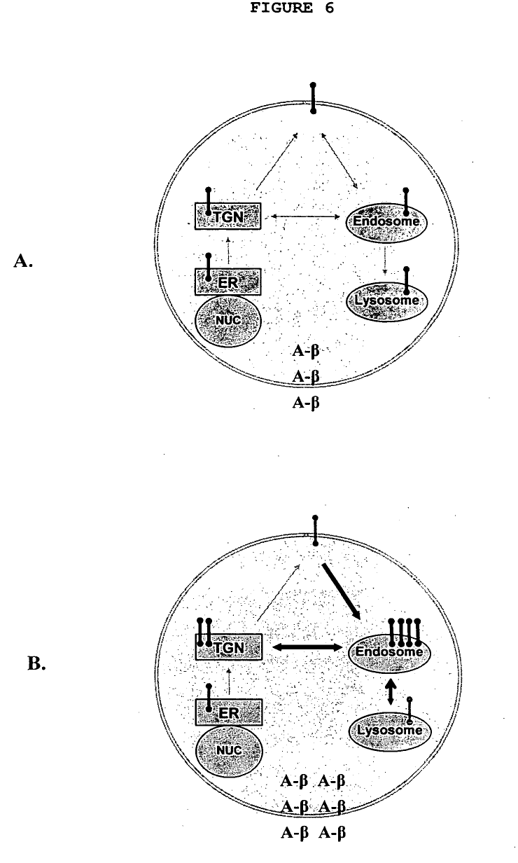

This study reveals a hypothesis-driven model based on the manner a molecule involved in AD pathogenesis should behave, both spatially and temporally. AD begins in the hippocampal formation. Each hippocampal subregion expresses a unique molecular profile, accounting for the reason the subregions are differentially vulnerable to mechanisms of dysfunction. Histological and brain imaging studies have shown that cells in the entorhinal cortex are most sensitive to AD, while cells in the dentate gyrus are relatively resistant to disease pathogenesis. Accordingly, the first assumption in the model was that, when comparing AD and control brains, a pathogenic molecule should be differentially expressed in the entorhinal cortex over the dentate gyrus (FIG. 1a). Imaging studies have further suggested that while the function of the entorhinal cortex is uniformly diminished in AD patients, entorhinal function is stable across the age-span among healthy controls. The model's second assumption, therefore, was that the temporal pattern of a pathogenic molecule should parallel the following observation: Expression levels should be reliably different when comparing between a group of AD patients and controls, but at the same time expression of the molecule should not differ across the age-span when assessing within a group of healthy subjects.

This model was applied to a microarray dataset the increased expression of a single molecule, VPS35, best distinguished sporadic AD from healthy controls. VPS35 is the core component of a sorting complex that traffics type I membrane-proteins, and acts to increase the concentration of membrane-proteins in the endosome and trans-golgi network (TGN). Although established in yeast, the full complement of membrane-proteins associated with VPS35 in humans remains unknown. To investigate this issue, the microarray dataset was examined and surprisingly showed that among all type I membrane-proteins, VPS35 expression was selectively associated with a sortilin-like receptor (SorLA) and with BACE-the enzyme which cleaves APP leading to a-β production (Hussain, Powell et al. 1999; Vassar, Bennett et al. 1999; Yan, Bienkowski et al. 1999). Indeed prior studies have established that BACE is actively transported to the TGN and endosome (Capell, Steiner et al. 2000; Huse, Pijak et al. 2000; Walter, Fluhrer et al. 2001), although the trafficking mechanisms responsible for this transport have remained unknown. Taken together with numerous reports showing that the activity of BACE is optimized within these intracellular organelles (Koo and Squazzo 1994; Xu, Sweeney et al. 1997; Perez, Soriano et al. 1999; Huse, Liu et al. 2002), these findings implicate membrane trafficking in sporadic AD and suggest a molecular mechanism for increased production of a-β peptide.

Materials and Methods

I. Human Brain Samples

Six Alzheimer's disease (AD) and 6 control brain samples were obtained at autopsy under a protocol approved by the institution's review board. The AD subjects included 4 females and 2 males with mean age of 86. The control subjects included 2 females and 4 males who ranged from 33-84 years old. Anatomic subregions were identified and sectioned using strict anatomical criteria following New York Brain Bank procedures. Samples were snap frozen in liquid nitrogen and stored at −80° C.

II. Gene-Expression Profiling

For each of the 12 brains, total RNA was extracted from entorhinal cortex and dentate gyrus tissue with TRIzol reagent (Invitrogen, Carlsbad, Calif.) and was purified with RNeasy column (Invitrogen). 10 μg total RNA were used to prepare double-stranded cDNA (Superscript, Invitrogen). The T7-(dT)₂₄ primer for cDNA synthesis contained a T7 RNA polymerase promoter site. An in vitro transcription reaction with biotin-labeled ribonucleotides was performed on the cDNA to produce cRNA probes (Bioarray High Yield RNA Transcript Labeling Kit, ENZO Life Sciences, Farmingdale, N.Y.). In the Gene Chip Facility of Columbia University, HG-U133A microarrays (GeneChip, Affymetrix, Santa Clara, Calif.) were hybridized with fragmented cRNA for 16 h in a 45° C. incubator with constant rotation at 60 g. Microarrays were washed and stained on a fluidics station, and scanned using a laser confocal microscope. HG-U133A microarrays were analyzed with Affymetrix Microarray Suite v5.0 and GeneSpring v5.0.3 (Silicon Genetics, Redwood City, Calif.) software, and scaled to a value of 500. Samples which had a 3′/5′ ratio of control genes actin and GAPDH greater than 7, were excluded from analysis. We excluded transcripts whose detection levels had a p-value greater than 0.05.

III. Real-Time Quantitative PCR

From the same samples that were used for microarray analysis, 2 μg total RNA and oligo(dT)₁₂₋₁₈ primer were used to generate single-stranded cDNA (Superscript, Invitrogen). The relative amount of VPS35 and Actin mRNAs were measured by real-time quantitative PCR using SmartCycler II (Cepheid, Sunnyvale, Calif.). The specific primer sets used were: VPS35 forward, 5′-CGAGAAGACCTCCCGAATCT-3′; VPS35 reverse, 5′-TCCGGAGTGCTGGGTAAAAC-3′; β-ACTIN forward, 5′-GATCATTGCTCCTCCTGAGC-3′; β-ACTIN reverse, 5′-GTCACCTTCACCGTTCCAGT-3′. The 25 μl reaction mixture was prepared following manufacturer's suggestion using 1 puReTaq Ready-To-Go PCR bead (Amersham, Amersham, UK), 50 mM MgCl₂, 1:10,000 SYBR Green (Molecular Probes, Eugene, Oreg.), 25 μM of each primer, and 800 ng cDNA. Following 60 s at 95° C., 40 cycles of 10 s at 95° C., 30 s at 66° C., 30 s at 72° C., and 20 s at 86° C. (80° C. for β-Actin reaction) were carried out. β-Actin expression was used for normalization.

IV. Human Brain Immunocytochemistry

Coronal blocks of human hippocampal formation were frozen-sectioned using a Microm cryostat at 8 μm thickness. Tissue was either directly quick-frozen or, in some cases, fixed 4% paraformaldehyde in PBS for 18 hr, cryoprotected in 25% sucrose in PBS, and then quick-frozen. Sections on slides were postfixed with 4% paraformaldehyde in PBS, washed with PBS, then treated with 3% H₂O₂, washed, and pre-incubated for 1 hr in Block solution consisting of 2% horse serum (Vector, Burlingame, Calif.), 1% bovine serum albumin (Sigma-Aldrich Chemical Co., St Louis, Mo.) and 0.1% Triton X-100 (Sigma) in PBS. Slides were then incubated 18 hr at 4° C. in diluted (1:500 in Block solution) rabbit polyclonal antiserum to VPS35p. This antiserum, directed to the yeast protein does react with the human protein by Western blot, and was kindly provided by C. Foote at the University of Missouri. After washing with PBS, immunoreactivity was detected by an avidin-biotin linked peroxidase method, using successive incubations and washes with goat anti-rabbit biotinylated IgG, Vectastain ABC-Elite reagent (Vector), and diaminobenzidine (Sigma) chromogen reagent. Sections were dehydrated and mounted using Permount (Fisher Scientific, Pittsburgh, Pa.).

V. Data Analysis

Variables from the microarray dataset that conformed to the spatial component of the model (FIG. 1a,b) were determined by performing a repeated-measures ANOVA, where the entorhinal cortex vs. dentate gyrus was included as the within-group factor, AD vs. controls was included as the between-group factor, and age was included as a covariate. The expression of 39 transcripts differed between AD and controls at a significant level of P<0.005. Among these, 35 variables did not conform to the temporal component of the model (FIG. 1c), determined by applying a repeated-measures ANOVA to the control group only. In order to identify which among the multiple variables best discriminated AD from controls, a stepwise logistic regression was performed. The restricted variables were included as covariates, and AD vs. control was included as the dependant variable. In order to confirm the VPS35 finding, a repeated measures ANOVA was also applied to the RT-PCR data.

Results

I. Expression of Four Genes Conformed to the Spatiotemporal Model of Alzheimer's Disease.

In order to identify the molecules that conform to the model, both the entorhinal cortex and the dentate gyrus (FIG. 1a) were isolated from 6 brains with pathologically proven AD, and from 6 brains free of pathology taken from healthy subjects who ranged from 33-84 years of age. A separate microarray analysis was performed on each tissue sample for a total of 24 microarray studies, and a repeated measures ANOVA, designed in accordance with the model, was applied to the dataset. By comparing the expression patterns of each individual's entorhinal cortex to their own dentate gyrus, and by comparing these differences between AD and controls, the spatial component of the model aided in constraining sources of noise, differences in genetic heritage and environmental exposures and regional differences in neuronal expression (FIG. 1b). The model's temporal component (FIG. 1c) was used to filter out potentially false-positive transcripts. The expression levels of 4 molecules conformed to both components of the model (FIG. 2): VPS35, which traffics membrane-proteins to intracellular organelles (Seaman, Marcusson et al. 1997; Pfeffer 2001); calnuc, which regulates calcium handling in the golgi (Lin, Le-Niculescu et al. 1998) and, COP9 and proteosome subunit IV, both involved in ubiquitin-based protein degradation (Glickman and Ciechanover 2002). Although serving diverse functions, the identified molecules are unified in playing a role in post-translational protein processing.

II. VPS35 is the Single Transcript that Best Discriminates Alzheimer's Disease from Controls.

The assumption that the expression of each gene is considered an independent event is easily violated in the intracellular milieu where molecular pathways and expression patterns influence each other. So, for example, an interaction between the molecular pathways of membrane-protein trafficking and ubiqitin-based degradation is well described (Katzmann, Odorizzi et al. 2002). In order to pinpoint, among the group of interconnected variables, the transcript most directly associated with AD, we applied a multivariate stepwise logistic regression to the restricted dataset. As expected, the regression analysis significantly discriminated AD from controls (chi-square=12.4; p=0.0004), but more importantly, VPS35 was isolated as the single variable underlying the effect. No other molecule significantly contributed to the analysis. Thus, the increase in VPS35 expression is the primary molecular difference discriminating sporadic AD and controls. Using real-time PCR, it was confirmed this effect showing that VPS35 mRNA is differentially elevated in the entorhinal cortex of AD brains (F=10.4; p=0.01) (FIG. 3a). A detailed exploration of the data, confirmed that the VPS35 effect, as detected with both microarray and RT-PCR, was caused by a differential increase in entorhinal cortex expression, and not by a differential decrease in dentate gyrus expression (FIG. 3c,d). Immunocytochemical staining showed that the translated protein of VPS35 is neuronal-predominant (FIG. 3b).

VPS35 is a vacuolar-sorting-protein (VPS) first described in yeast (Paravicini, Horazdovsky et al. 1992) as part of a systematic search for molecules that govern trafficking among intracellular organelles. Recently, the ortholog of VPS35 has been identified in mammalian species, including in humans in whom VPS35 is highly expressed in the brain, and localizes to the endosome and TGN (Edgar and Polak 2000; Haft, de la Luz Sierra et al. 2000). VPS35 is the molecular core to which other sorting molecules bind (Seaman, Marcusson et al. 1997; Seaman, McCaffery et al. 1998; Nothwehr, Ha et al. 2000)—such as the sorting nexins (Edgar and Polak 2000; Haft, de la Luz Sierra et al. 2000)-and together form a sorting complex that traffics type I membrane-proteins. VPS35 and the sorting complex act to increase the concentration of type I membrane-receptors in the endosome and TGN-either by decreasing lysosomal degradation (Seaman, Marcusson et al. 1997; Seaman, McCaffery et al. 1998) or by increasing cell surface internalization (Kurten, Cadena et al. 1996; Kurten, Eddington et al. 2001)

III. VPS35 is Selectively Associated with BACE and a Sortilin-Like Receptor in the Human Hippocampal Formation.

In yeast, VPS10 is the dominant type I membrane-protein associated with VPS35 (Seaman, Marcusson et al. 1997). Although sortilin and members of the sortilin-like family (Hampe, Rezgaoui et al. 2001), membrane-proteins that contain VPS10-domains, are suspected targets in animal cells, the full complement of human membrane-proteins associated with VPS35 remains unknown (Edgar and Polak 2000; Haft, de la Luz Sierra et al. 2000; Pfeffer 2001). In an effort to identify candidate proteins, the microarray dataset was searched for an association between VPS35 and any type I membrane-protein in the entorhinal cortex of AD brains. Surprisingly, the expression of only two molecules were found to correlate with VPS35 expression: BACE (beta=0.93; p=0.008) and a sortilin-like receptor (SorLA) (beta=0.84; p=0.038) (FIG. 4a,b). The association between VPS35 and BACE (beta=0.58; p=0.003) and between VPS35 and SorLA (beta=0.76; p=0.0002) was maintained when we extended the analysis to include all subjects and both hippocampal subregions (FIG. 4a,b). Importantly, the expression of BACE and SorLA were not associated with each other (beta=0.32; p=0.113), showing that each membrane-protein was independently associated with VPS35 in the human hippocampal formation. Although prior studies predict an association between VPS35 and SorLA (Seaman, Marcusson et al. 1997; Hampe, Rezgaoui et al. 2001), the observed association between VPS35 and BACE in the human hippocampal formation was unexpected. If VPS35 plays a role in BACE trafficking, then, as with other membrane-proteins, increased VPS35 expression is expected to cause an accumulation of BACE within the endosome and TGN.

IV. Neither BACE nor APP Conforms to the Spatial Model of Alzheimer's Disease.

Because BACE and the substrate upon which this enzyme works, namely APP, play essential roles in AD pathophysiology, it was important to directly test whether these molecules were differentially expressed in the entorhinal cortex of AD brains. It was possible, for example, that the temporal component of the model was overly stringent and that these molecules were unnecessarily filtered; or, that their expression differences trended toward significance, but because of power issues did not survive the statistical cut-off. In fact, on direct testing we found that neither the expression levels of BACE (F=0.002; p=0.97) nor the expression levels of APP (F=0.017; p=0.90) differed, even subtly, between AD and controls (FIG. 5). We also tested SorLA, and found that its expression did not significantly differ between the groups (F=0.032; p=0.98) (FIG. 5). Thus, differential expression of BACE or APP cannot account for selective neuronal vulnerability in AD pathophysiology. Moreover, selective vulnerability appears unrelated to differential mechanisms of membrane trafficking, as VPS35 was associated with BACE in both subregions and in all subjects. Discussion

By relying on an assumption-driven model, the analysis identified only a limited number of molecules that significantly distinguished sporadic AD from controls. The assumptions of the model might have been too stringent, potentially filtering out expression differences that were true and not false positives. Therefore, we cannot state with confidence that molecules not isolated by our analysis are not relevant to sporadic AD, unless specifically tested as in the case of BACE and APP. Nevertheless, this heightened specificity increases the confidence that the four identified molecules are associated with sporadic AD.

Among the many proposed theories, the molecules isolated suggest that post-translational protein processing might play a pivotal role in disease pathogenesis. More precisely, by identifying an increase in VPS35 expression as the primary difference between sporadic AD and healthy controls, a specific processing mechanism is highlighted-the trafficking of membrane-proteins into intracellular organelles. VPS35 is not only the core to which the other molecules of the sorting complex bind, but it itself is the ‘receptor’ of the sorting complex that identifies and binds the to-be trafficked membrane-protein (Seaman, McCaffery et al. 1998; Edgar and Polak 2000; Haft, de la Luz Sierra et al. 2000).

VPS35 was first described in yeast, and by systematically manipulating VPS35 expression the cargo proteins that VPS35 binds and the pathway that it traffics have been determined (Paravicini, Horazdovsky et al. 1992; Seaman, Marcusson et al. 1997). In yeast, VPS35 traffics type I membrane-proteins from the endosome back to the TGN, and because of the direction of this trafficking pathway the yeast sorting complex has been called the retromer (Seaman, McCaffery et al. 1998). This trafficking pathway salvages membrane-proteins from lysosomal degradation, and therefore VPS35 acts to increase the concentration of targeted membrane-proteins within the endosome and TGN (Seaman, Marcusson et al. 1997).

The mammalian ortholog of VPS35 (Edgar and Polak 2000; Haft, de la Luz Sierra et al. 2000), and the orthologs of some of the other molecules of the sorting complex-notably, sorting nexin 1 (SNX1) and sorting nexin 2 (SNX2)—have been identified (Horazdovsky, Davies et al. 1997; Haft, de la Luz Sierra et al. 1998). In humans, VPS35 is dominantly expressed in the brain, and is localized to the endosome and TGN (Edgar and Polak 2000; Haft, de la Luz Sierra et al. 2000).

The structural similarities between the yeast and mammalian sorting molecules suggest a preservation of function. Indeed, as in yeast, mammalian VPS35 is the core molecule to which the sorting nexins bind, forming a mammalian sorting complex that also traffics type I membrane-proteins (Haft, de la Luz Sierra et al. 2000). Studies directly manipulating mammalian VPS35 expression have not yet been performed. Therefore, despite overall similarities in structure and function many features of mammalian VPS35 remain unclear. As in yeast, mammalian VPS35 may traffic membrane-proteins between the endosome and TGN (Pfeffer 2001), in which case mammalian VPS35 will act to reduce lysosomal degradation, thereby increasing the concentration of membrane-proteins within these intracellular organelles. Nevertheless, studies manipulating the expression of sorting nexins suggest that an alternative pathway needs to be considered. SNX1 and SNX2 have been shown to traffic type I membrane-proteins from the cell surface to the endosome (Worby and Dixon 2002), and because VPS35 is intimately related to these sorting nexins (Haft, de la Luz Sierra et al. 2000), mammalian VPS35 might also traffic along the same surface-to-endosome pathway. Overexpressing the sorting nexins increases the endosomal concentration of surface membrane-proteins (Kurten, Cadena et al. 1996), from where the protein can be secondarily trafficked back to the TGN (Worby and Dixon 2002). If VPS35 indeed works in parallel to the sorting nexins, it is predicted that overexpressing VPS35 should likewise increase the concentration of the trafficked membrane-protein within these intracellular organelles. Thus, whether mammalian VPS35 salvages membrane-proteins from lysosomal degradation or whether it accelerates internalization of membrane-proteins from the cell surface, overexpressing mammalian VPS35 is expected to increase the concentration of type I membrane-proteins in the endosome and TGN.

The cargo membrane-proteins to which VPS35 binds have also been more fully investigated in yeast, in which VPS10 appears to be the most common type I membrane-protein trafficked by the VPS35-based sorting complex (Seaman, Marcusson et al. 1997; Seaman, McCaffery et al. 1998). Many type I membrane-proteins are proposed candidate targets of mammalian VPS35—including sortilin and the family of sortilin-like receptors which contain VSP10-domains (Hampe, Rezgaoui et al. 2001), the range of surface receptors internalized by the sorting nexins (Worby and Dixon 2002), and a host of other membrane-proteins that are trafficked between the endosome and TGN (Pfeffer 2001). In the absence of studies directly manipulating mammalian VPS35 expression, however, its primary targets remain unknown.

In attempting to identify candidate targets, BACE was unexpectedly found and is one of only two type I membrane-proteins in the human hippocampal formation to tightly correlate with VPS35 expression. Since all sortilin-like receptors contain a VPS10 domain, a known target of VPS35 in yeast, and since these membrane-proteins are actively trafficked among the TGN, cell surface, and endosome, the association between VPS35 and SorLA was less surprising. Recent studies elucidating the post-translation processing of BACE, (Capell, Steiner et al. 2000; Huse, Pijak et al. 2000; Walter, Fluhrer et al. 2001), have shown that it too is actively trafficked among the TGN, cell surface, and endosome (FIG. 6a), although the identity of the trafficking molecules remain unknown (Huse, Pijak et al. 2000). BACE shares structural and functional similarities with VPS10, containing receptors and with other membrane-proteins implicated with VPS35, such as the mannose-6-phosphate receptor (Pfeffer 2001; He, Chang et al. 2002). Therefore VPS35 and the mammalian sorting complex must be considered candidates for BACE trafficking.

Nevertheless, the observed correlation between the expression of VPS35 and BACE only establishes an association between these molecules, and elucidating the precise biochemical nature underlying this association requires an in depth analysis in cell culture preparations. At this point, however, a parsimonious and plausible interpretation of the data is that VPS35 plays some undefined role in BACE trafficking. In any case, the association between VPS35 and BACE suggests a model that can account for regionally selective increases in a-β production (FIG. 6b) . Increasing the expression of VPS35 will increase the concentration of BACE in the endosome and TGN-either by salvaging BACE from the lysosomal pathway, or by accelerating its internalization from the cell surface.

Numerous studies have established that the endosome and the TGN are the main sites where BACE cleaves APP (Koo and Squazzo 1994; Xu, Sweeney et al. 1997; Perez, Soriano et al. 1999; Huse, Liu et al. 2002), and thus the end result of VPS35 overexpression, as observed in the entorhinal cortex of AD brains, is the increased production of a-β peptide. By implicating membrane trafficking, this model reconciles the dilemma of how production of a-β peptide in the entorhinal cortex (Lue, Kuo et al. 1999) can increase in the absence of polymorphisms in, or overexpression of, BACE or APP. The redistribution of BACE throughout the different cellular compartments-the proposed effect of VPS35 overexpression in the entorhinal cortex, should be enough to alter the rate of a-β production, and therefore neither a quantitative nor qualitative alteration in BACE is required.

### REFERENCES

1. Benjannet, S. et al., Post-translational processing of beta-secretase (beta-amyloid-converting enzyme) and its ectodomain shedding. The pro- and transmembrane/cytosolic domains affect its cellular activity and amyloid-beta production, (2001) J Biol Chem 276(14): p. 10879-87.
2. Braak, H. et al., Evolution of the neuropathology of Alzheimer's disease, (1996) Acta Neurol Scand Suppl 165: p.3-12.
3. Capell, A. et al., Maturation and pro-peptide cleavage of beta-secretase, (2000) J Biol Chem 275(40): p. 30849-54.
4. de Leon, M. et al., Prediction of cognitive decline in normal elderly subjects with 2-[(18)F]fluoro-2-deoxy-D-glucose/poitron-emission tomography (FDG/PET), (2001) Proc Natl Acad Sci USA 98(19): p. 10966-71.
5. Edgar, A. J. et al., Human homologues of yeast vacuolar protein sorting 29 and 35, (2000) Biochem Biophys Res Commun 277(3): p. 622-30.
6. Glickman, M. H. and Ciechanover A., The ubiquitin-proteasome proteolytic pathway: destruction for the sake of construction, (2000) Physiol Rev 82(2): p. 373-428.
7. Gomez-Isla, T. et al. Profound loss of layer II entorhinal cortex neurons occurs in very mild Alzheimer's disease, (1996) J Neurosci 16(14): p. 4491-500.
8. Gu, C. C. et al., Role of gene expression microarray analysis in finding complex disease genes, (2002) Genet Epidemiol 23(1): p. 37-56.
9. Haft, C. R. et al., Human orthologs of yeast vacuolar protein sorting proteins Vps26, 29, and 35: assembly into multimeric complexes, (2000) Mol Biol Cell 11(12): p. 4105-16.
10. Haft, C. et al., Identification of a family of sorting nexin molecules and characterization of their association with receptors, (1998) Mol Cell Biol 18(12): p. 7278-87.
11. Hampe, W. et al., The genes for the human VPS10 domain-containing receptors are large and contain many small exons, (2001) Hum Genet 108(6): p. 529-36.
12. Hardy, J. et al., The amyloid hypothesis of Alzheimer's disease: progress and problems on the road to therapeutics, (2002) Science 297(5580): 353-6.
13. Harrison, P. et al., The relative importance of premortem acidosis and postmortem interval for human brain gene expression studies: selective mRNA vulnerability and comparison with their encoded proteins, (1995) Neurosci Lett 200(3): p. 151-4.
14. He, X. et al., Memapsin 2 (beta-secretase) cytosolic domain binds to the VHS domains of GGA1 and GGA2: implications on the endocytosis mechanism of memapsin 2, (2002) FEBS Lett 524(1-3): p.183-7.
15. Horazdovsky, B. F. et al. A sorting nexin-1 homologue, Vps5p, forms a complex with Vps17p and is required for recycling the vacuolar protein-sorting receptor, (1997) Mol Biol Cell 8(8):p. 1529-41.
16. Huse, J. T. et al., Beta-secretase processing in the trans-Golgi network preferentially generates truncated amyloid species that accumulate in Alzheimer's disease brain, (2002) J Biol Chem 277(18):p. 16278-84.
17. Huse, J. T. et al., Maturation and endosomal targeting of beta-site amyloid precursor protein-cleaving enzyme. The Alzheimer's disease beta-secretase, (2000) J Biol Chem 275(43): p. 33729-37.
18. Hussain, I. et al., Identification of a novel aspartic protease (Asp 2) as beta-secretase, (1999) Mol Cell Neurosci 14(6): p. 419-27.
19. Jacobs, D. et al., Neuropsychological detection and characterization of preclinical Alzheimer's disease, (1995) Neurology 45(5): p. 957-62.
20. Jacobsen, L. et al., Activation and functional characterization of the mosaic receptor SorLA/LR11, (2001) J Biol Chem 276(25): p. 22788-96.
21. Katzmann, D. J. et al., Receptor downregulation and multivesicular-body sorting, (2002) Nat Rev Mol Cell Biol 3(12): p. 893-905.
22. Koo, E. H. et al., Evidence that production and release of amyloid beta-protein involves the endocytic pathway, (1994) J Biol Chem 269(26): p. 17386-9.
23. Kurten, R. C. et al., Enhanced degradation of EGF receptors by a sorting nexin, SNX1., (1996) Science 272(5264): p. 1008-10.
24. Kurten, R. C. et al., Self-assembly and binding of a sorting nexin to sorting endosomes, (2001) J Cell Sci 114(Pt 9): p. 1743-56.
25. Lin, P. et al., The mammalian calcium-binding protein, nucleobindin (CALNUC), is a Golgi resident protein, (1998) J Cell Biol 141(7): p. 1515-27.
26. Lue, L. F. et al., Soluble amyloid beta peptide concentration as a predictor of synaptic change in Alzheimer's disease, (1999) Am J Pathol 155(3): p. 853-62.
27. McLean, C. A. et al., Soluble pool of Abeta amyloid as a determinant of severity of neurodegeneration in Alzheimer's disease, (1999) Ann Neurol 46(6): p. 860-6.
28. Naslund, J. et al., Correlation between elevated levels of amyloid beta-peptide in the brain and cognitive decline, (2000) Jama 283(12): p. 1571-7.
29. Nothwehr, S. F. et al., Sorting of yeast membrane proteins into an endosome-to-Golgi pathway involves direct interaction of their cytosolic domains with Vps35p, (2000) J Cell Biol 151(2): p. 297-310.
30. Paravicini, G. et al., Alternative pathways for the sorting of soluble vacuolar proteins in yeast: a vps35 null mutant missorts and secretes only a subset of vacuolar hydrolases, (1992) Mol Biol Cell 3(4): p. 415-27.
31. Perez, R. G. et al., Mutagenesis identifies new signals for beta-amyloid precursor protein endocytosis, turnover, and the generation of secreted fragments, including Abeta42, (1999) J Biol Chem 274(27): 18851-6.
32. Pfeffer, S. R., Membrane transport: retromer to the rescue, (2001) Curr Biol 11(3): p. R109-11.
33. Pongrac, J., Middleton F. A. et al., Gene expression profiling with DNA microarrays: advancing our understanding of psychiatric disorders, (2002) Neurochem Res 27(10): p. 1049-63.
34. Seaman, M. N. et al., Endosome to Golgi retrieval of the vacuolar protein sorting receptor, Vps10p, requires the function of the VPS29, VPS30, and VPS35 gene products, (1997) J Cell Biol 137(1): p. 79-92.
35. Seaman, M. N. et al. A membrane coat complex essential for endosome-to-Golgi retrograde transport in yeast, (1998) J Cell Biol 142(3): p. 665-81.
36. Selkoe, D. J. et al., Deciphering the genetic basis of Alzheimer's disease, (2002) Annu Rev Genomics Hum Genet 3: p. 67-99.
37. Slonim, D. K., From patterns to pathways: gene expression data analysis comes of age, (2002) Nat Genet 32 Suppl: p. 502-8.
38. Small, S. A., Age-related memory decline; current concepts and future directions, (2001) Archives of Neurology 58: 360-364.
39. Small, S. A., et al., Differential regional dysfunction of the hippocampal formation among elderly with memory decline and Alzheimer's disease, (1999) Ann Neurol 45(4): p. 466-72.
40. Small, S. A. et al., Imaging hippocampal function across the human life span: is memory decline normal or not?, (2002) Ann Neurol 51(3): p. 290-5.
41. Vassar, R. et al., Beta-secretase cleavage of Alzheimer's amyloid precursor protein by the transmembrane aspartic protease BACE, (1999) Science 286(5440): p. 735-41.
42. Walter, J. et al., Phosphorylation regulates intracellular trafficking of beta-secretase, (2001) J Biol Chem 276(18): p. 14634-41.
43. Wang, J. et al., The levels of soluble versus insoluble brain Abeta distinguish Alzheimer's disease from normal and pathologic aging, (1999) Exp Neurol 158(2):p. 328-37.
44. West, M. J. et al. Differences in the pattern of hippocampal neuronal loss in normal ageing and Alzheimer's disease, (1994) Lancet 344(8925): p. 769-72.
45. Worby, C. A. et al., Sorting out the cellular functions of sorting nexins, (2002) Nat Rev Mol Cell Biol 3(12): p. 919-31.
46. Wurmbach, E. et al., Validated genomic approach to study differentially expressed genes in complex tissues, (2002) Neurochem Res 27(10): p. 1027-33.
47. Xu, H., Sweeney D. et al., Generation of Alzheimer beta-amyloid protein in the trans-Golgi network in the apparent absence of vesicle formation, (1997) Proc Natl Acad Sci USA 94(8): p. 3748-52.
48. Yan, R. et al., Membrane-anchored aspartyl protease with Alzheimer's disease beta-secretase activity, (1999) Nature 402(6761): p. 533-7.
49. Zhao, X. et al., Transcriptional profiling reveals strict boundaries between hippocampal subregions, (2001) J Comp Neurol 441(3): p. 187-96.

1. A method for determining whether an agent causes a reduction in the expression of a retromer complex protein, comprising the steps of:
(a) contacting the agent with a eukaryotic cell under conditions which, in the absence of the agent, permit expression of the retromer complex protein;
(b) after a suitable period of time, determining the amount of expression in the cell of the retromer complex protein; and
(c) comparing the amount of expression determined in step (b) with the amount of expression which occurs in the absence of the agent, whereby a reduced amount of expression in the presence of the agent indicates that the agent causes a reduction in the expression of the retromer complex protein.

2. The method of claim 1, wherein the retromer complex protein is VPS35.

3. The method of claim 1, wherein the retromer complex protein is selected from the group consisting of VPS17, VPS26, VPS29, sorting nexin 1 and sorting nexin 2.

4. The method of claim 1, wherein the cell is present in a cell culture.

5. The method of claim 1, wherein the cell is a brain cell.

6. The method of claim 1, wherein determining the amount of expression is performed by determining the amount of retromer complex protein-encoding mRNA in the cell.

7. The method of claim 1, wherein determining the amount of expression is performed by determining the amount of retromer complex protein in the cell.

8. The method of claim 7, wherein determining the amount of retromer complex protein in the cell is performed using an antibody specific for such protein.

9. A method for determining whether an agent causes a reduction in the activity of a retromer complex, comprising the steps of:
(a) contacting the agent with a eukaryotic cell under conditions which, in the absence of the agent, permit activity of the retromer complex;
(b) determining the amount of activity in the cell of the retromer complex; and
(c) comparing the amount of activity determined in step (b) with the amount of activity which occurs in the absence of the agent, whereby a reduced amount of activity in the presence of the agent indicates that the agent causes a reduction in the activity of the retromer complex.

10. The method of claim 9, wherein the retromer complex comprises the protein VPS35.

11. The method of claim 9, wherein the retromer complex comprises a protein selected from the group consisting of VPS17, VPS26, VPS29, sorting nexin 1 and sorting nexin 2.

12. The method of claim 9, wherein the cell is present in a cell culture.

13. The method of claim 9, wherein the cell is a brain cell.

14. A method for reducing the expression of a retromer complex protein in a cell comprising introducing into the cell an agent which specifically interferes with the expression of the retromer complex protein in the cell.

15. The method of claim 14, wherein the retromer complex protein is VPS35.

16. The method of claim 14, wherein the retromer complex protein is selected from the group consisting of VPS17, VPS26, VPS29, sorting nexin 1 and sorting nexin 2.

17. The method of claim 14, wherein the cell is present in a cell culture.

18. The method of claim 14, wherein the cell is a brain cell.

19. The method of claim 14, wherein the agent is a nucleic acid.

20. The method of claim 19, wherein the nucleic acid is a small interfering RNA.

21. The method of claim 19, wherein the nucleic acid is a ribozyme.

22. The method of claim 19, wherein the nucleic acid is a DNAzyme.

23. The method of claim 19, wherein the nucleic acid is an antisense molecule.

24. A method for treating a subject afflicted with Alzheimer's disease comprising administering to the subject a therapeutically effective amount of an agent which specifically interferes with the expression of the retromer complex protein in the cells of the subject's brain which express a-β peptide.

25. The method of claim 24, wherein the retromer complex protein is VPS35.

26. The method of claim 24, wherein the retromer complex protein is selected from the group consisting of VPS17, VPS26, VPS29, sorting nexin 1 and sorting nexin 2.

27. The method of claim 24, wherein the agent is a nucleic acid.

28. The method of claim 27, wherein the nucleic acid is a small interfering RNA.

29. The method of claim 27, wherein the nucleic acid is a ribozyme.

30. The method of claim 27, wherein the nucleic acid is a DNAzyme.

31. The method of claim 27, wherein the nucleic acid is an antisense molecule.

32. A pharmaceutical composition comprising:
(a) an agent which specifically interferes with the expression of a retromer complex protein when introduced into a cell; and
(b) a pharmaceutically acceptable carrier.

33. An article of manufacture comprising:
(a) a packaging material having therein an agent which specifically interferes with the expression of a retromer complex protein when introduced into a cell; and
(b) a label indicating a use for the agent in treating a subject afflicted with Alzheimer's disease.

34. A method for identifying a potential pathogenic nucleic acid transcript with respect to a brain disorder comprising the steps of:
(a) contacting (i) a brain nucleic acid microarray with (ii) a nucleic acid sample from afflicted brain tissue, wherein the brain tissue is from a brain region expected to contain the pathogenic nucleic acid transcript, under conditions permitting nucleic acid hybridization;
(b) determining the hybridization pattern resulting from step (a); and
(c) comparing the hybridization pattern so determined with the hybridization pattern determined upon separately contacting the microarray with a nucleic acid sample from (i) afflicted brain tissue which is not from a brain region expected to contain the pathogenic nucleic acid transcript, (ii) non-afflicted brain tissue which is from a brain region expected to contain the pathogenic nucleic acid transcript, and (iii) non-afflicted brain tissue which is not from a brain region expected to contain the pathogenic nucleic acid transcript,
wherein if a nucleic acid from the sample of step (a) is present in a greater amount at a particular locus in the hybridization pattern of step (b) than at that locus in any of the hybridization patterns in step (c), or is present in a lesser amount at a particular locus in the hybridization pattern of step (b) than at that locus in any of the hybridization patterns in step (c), it is a potential pathogenic nucleic acid transcript.

35. The method of claim 34, further comprising the step of determining whether the increase or decrease, as applicable, in the amount of potential pathogenic nucleic acid transcript so identified is concurrent with the onset of the brain disorder in an afflicted subject.

36. The method of claim 34, wherein the subject is human.

37. The method of claim 34, wherein the brain disorder is Alzheimer'disease.


SEQUENCE LISTING (10 sequences)

SEQ ID NO: 1 - DNA, 21 bp, artificial
  siRNA Target Sequence
aagtggcagatctctacgaac

SEQ ID NO: 2 - DNA, 21 bp, artificial
  siRNA
guggcagaucucuacgaactt

SEQ ID NO: 3 - DNA, 21 bp, artificial
  siRNA
guucguagagaucugccactt

SEQ ID NO: 4 - DNA, 21 bp, artificial
  siRNA Target Sequence
aagcacagctagctgccatca

SEQ ID NO: 5 - DNA, 21 bp, artificial
  siRNA
gcacagcuagcugccaucatt

SEQ ID NO: 6 - DNA, 21 bp, artificial
  siRNA
ugauggcagcuagcugugctt

SEQ ID NO: 7 - DNA, 20 bp, artificial
  PCR primer
cgagaagacctcccgaatct

SEQ ID NO: 8 - DNA, 20 bp, artificial
  PCR primer
tccggagtgctgggtaaaac

SEQ ID NO: 9 - DNA, 20 bp, artificial
  PCR primer
gatcattgctcctcctgagc

SEQ ID NO: 10 - DNA, 20 bp, artificial
  PCR primer
gtcaccttcaccgttccagt